(19) United States
(12) Patent Application Publication
(10) Pub. No.: US 2024/0170133 A1
(43) Pub. Date: May 23, 2024

(54) Image-Based Severity Detection Method and System

(71) Applicant: Georgia Tech Research Corporation, Atlanta, GA (US)

(72) Inventors: Ghassan AlRegib, Atlanta, GA (US); Kiran Kokilepersaud, Atlanta, GA (US); Mohit Prabhushankar, Atlanta, GA (US)

(21) Appl. No.: 18/513,805

(22) Filed: Nov. 20, 2023

Related U.S. Application Data
(60) Provisional application No. 63/426,489, filed on Nov. 18, 2022.

(51) Int. Cl. G16H 30/40; G16H 30/20; G16H 50/20

(52) U.S. Cl. CPC G16H 30/40; G16H 30/20; G16H 50/20

(57) ABSTRACT

An exemplary system and method for contrastive learning that can generate pseudo severity-based labels for unlabeled medical images using gradient measures from an anomaly detection operation. The severity labels can be then used for diagnosis of a disease or medical condition or as labels for as a training data set for training of another machine learning model. The training can be performed in combination with biomarker data.

### RELATED APPLICATION

This US patent application claims priority to, and the benefit of, U.S. Provisional Patent Application No. 63/426,489, filed Nov. 18, 2022, which is incorporated by reference herein in its entirety.

### BACKGROUND

Deep learning is the subset of machine learning methods that are based on artificial neural networks with representation learning. Deep learning approaches generally rely on access to a large quantity that are labeled. Labeled data can be used as ground truth during the training operation and are generally provided in a supervised manner by radiologists and other specialists for medical diagnostics or imaging.

The dependence of training of deep learning systems on potentially expensive labels makes them sub-optimal for the various constraints of the medical field.

There is a benefit to improving deep learning systems.

### SUMMARY

An exemplary system and method are disclosed for contrastive learning that can generate pseudo-severity-based labels for unlabeled medical images using gradient measures from an anomaly detection operation. The anomaly detection operation can generate anomaly scores with respect to a trained model that has learned the healthy or baseline distribution and the degree a dataset is anomalous to the healthy/baseline distribution. Example statistics or parameters that capture the severity of samples, as an anomaly from the healthy/baseline data set, include the reconstruction error, gradient response induced by a sample, and 11-norm of a latent space vector. Progressively more anomalous samples would represent samples with greater severity and these scores would be a quantification of severity. The severity labels can be then used for the diagnosis of a disease or medical condition or as labels for a training data set for training of another machine learning model. The training can be performed in combination with biomarker data. A study was conducted to develop contrastive learning operations that can generate pseudo-severity-based labels for unlabeled optical coherence tomography (OCT) medical images. The study observed 6% improved biomarker classification accuracy for Diabetic Retinopathy.

The exemplary system and method may be employed to develop trained machine learning models for any number of imaging modalities, for example, optical coherence tomography, ultrasound, magnetic resonance imaging, and computing tomography, among other modalities described or referenced herein.

In an aspect, a method is disclosed of training a machine learning model, the method comprising: in a contrastive learning operation, training a baseline ML model via a first data set, the first data set consisting only of data for a non-anomalous, normal, or healthy set (e.g., patient, sample, etc.); in the contrastive learning operation, generating gradient severity score vector from the baseline ML model for a second data set, the second data set comprising data for anomalous or unhealthy set, wherein the second data set is unlabeled with respect to severity; and in the contrastive learning operation, tiering the severity score vector into a plurality of severity classes, including a first severity class associated with a first severity score label and a second severity class associated with a second severity score label (e.g., wherein the first severity score label is an indication of a presence or severity of disease or condition), wherein at least one of the first severity score label and the second severity score label is used (i) for diagnosis or (ii) as labels for the second data set as a training data set for a second ML model or the baseline ML model.

In some embodiments, the step of tiering the severity score vector into the plurality of severity classes comprises: ordering the severity score vector by rank to generate a ranked list of vector elements of the severity score vector; and arranging the ranked list of vector elements of the severity score vector into a plurality of bins, wherein a first bin corresponds to the first severity class, and wherein the second bin corresponds to the second severity class.

In some embodiments, the method further includes selecting a portion of the second data set based on the gradient labels (e.g., the first severity score label or the second severity score label); and training the second ML model or the baseline ML model via the selected portion of the second data set.

In some embodiments, the second data set comprises candidate biomarker data for anomalous or unhealthy set, and wherein the method further comprising: training the second ML model or the baseline ML model via the second data set, wherein the gradient labels are used as ground truth for a set of biomarkers identified in the second data set.

In some embodiments, the method further includes outputting, via a report or display, the respective gradient label and classifier output of the baseline ML model, wherein the respective gradient label and classifier output is used for the diagnosis of a disease or a medical condition.

In some embodiments, the first data set comprises image data from a medical scan.

In some embodiments, the first data set comprises image data from a sensor.

In some embodiments, the baseline ML model comprises an auto-encoder.

In some embodiments, the candidate biomarker data includes at least one of: Intraretinal Fluid (IRF), Diabetic Macular Edema (DME), and Intra-Retinal Hyper-Reflective Foci (IRHRF).

In another aspect, a method is disclosed comprising: receiving a data set; determining, via a trained machine learning model, a presence or severity value associated with a disease or medical condition using the data set; outputting, via a report or graphical user interface, the determined presence or severity value, wherein the trained machine learning model was trained in a contrastive learning operation, the contrastive learning operation comprising training a baseline ML model via a first training data set, the first training data set consisting of data for a non-anomalous, normal, or healthy set (e.g., patient, sample, etc.); generating gradient severity score vector from the baseline ML model for a second training data set, the second training data set comprising candidate biomarker data for anomalous or unhealthy set, wherein the second training data set is unlabeled with respect to severity; tiering the severity score vector into a plurality of severity classes, including a first severity class associated with a first severity score label and a second severity class associated with a second severity score label (e.g., wherein the first severity score label is an indication of a presence or severity of disease or condition); and generating the trained machine learning model using the first severity score label and the second severity score label.

In some embodiments, the step of tiering the severity score vector into the plurality of severity classes comprises: ordering the severity score vector by rank to generate a ranked list of vector elements of the severity score vector; and arranging the ranked list of vector elements of the severity score vector into a plurality of bins, wherein a first bin corresponds to the first severity class, and wherein the second bin corresponds to the second severity class.

In some embodiments, the second data set comprises candidate biomarker data for anomalous or unhealthy set, wherein the method to train the machine learning model further comprises training the second ML model or the baseline ML model via the second data set, wherein the gradient labels are used as ground truth for a set of biomarkers identified in the second data set.

In some embodiments, the first training data set comprises image data from a medical scan.

In some embodiments, the first training data set comprises image data from a sensor.

In some embodiments, the baseline ML model comprises an auto-encoder.

In another aspect, a system is disclosed comprising: a processor; and a memory having instructions stored thereon, wherein execution of the instructions by the processor causes the processor to: receive a data set; determine, via a trained machine learning model, a presence or severity value associated with a disease or medical condition using the data set; and output, via a report or graphical user interface, the determined presence or severity value, wherein the trained machine learning model was trained in a contrastive learning operation, the contrastive learning operation comprising: training a baseline ML model via a first training data set, the first training data set consisting of data for a non-anomalous, normal, or healthy set (e.g., patient, sample, etc.); generating gradient severity score vector from the baseline ML model for a second training data set, the second training data set comprising candidate biomarker data for anomalous or unhealthy set, wherein the second training data set is unlabeled with respect to severity; tiering the severity score vector into a plurality of severity classes, including a first severity class associated with a first severity score label and a second severity class associated with a second severity score label (e.g., wherein the first severity score label is an indication of a presence or severity of disease or condition); and generating the trained machine learning model using the first severity score label and the second severity score label.

As used herein, a processor is a processing unit configured via computer-readable instructions or comprising digital circuitries to execute instructions. A processor can include one or more microprocessors, FPGAs, ASICs, AI processors, or combinations or cores thereof.

In some embodiments, the instructions to tier the severity score vector into the plurality of severity classes comprises: instructions to order the severity score vector by rank to generate a ranked list of vector elements of the severity score vector; and instructions to arrange the ranked list of vector elements of the severity score vector into a plurality of bins, wherein a first bin corresponds to the first severity class, and wherein the second bin corresponds to the second severity class.

In some embodiments, the first training data set comprises image data from a medical scan.

In some embodiments, the first training data set comprises image data from a sensor.

In some embodiments, the baseline ML model comprises an auto-encoder.

The terms “treat,” “treating,” “treatment,” and grammatical variations thereof as used herein include partially or completely delaying, alleviating, mitigating, or reducing the intensity of one or more attendant symptoms of a disorder or condition and/or alleviating, mitigating or impeding one or more causes of a disorder or condition. Treatments according to the disclosure may be applied preventively, prophylactically, palliatively, or remedially. Treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of cancer.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue, and most invade surrounding tissues, can metastasize to several sites, are likely to recur after attempted removal, and may cause the death of the patient unless adequately treated. As used herein, the term neoplasia is used to describe all cancerous disease states and embraces or encompasses the pathological process associated with malignant, hematogenous, ascitic, and solid tumors. The cancers that may be identified and diagnosed by the devices and methods disclosed herein may comprise carcinomas, sarcomas, lymphomas, leukemias, germ cell tumors, or blastomas.

Further representative cancers include, but are not limited to, bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers; breast cancers; endocrine system cancers; eye cancers; gastrointestinal cancers; genitourinary and gynecologic cancers; head and neck cancers; hematopoietic cancers; thoracic and respiratory cancers; HIV/AIDS-related cancers; desmoplastic small round cell tumor; and liposarcoma.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 shows an example system configured with a severity analysis module to perform contrastive learning to generate pseudo-severity-based labels for unlabeled data set and train a classification engine in accordance with an illustrative embodiment.

FIGS. 2A, 2B, 2C each shows an example method to perform contrastive learning to generate pseudo-severity-based labels for unlabeled data to train a classification engine in accordance with an illustrative embodiment.

FIG. 3 shows example biomarkers in OCT scans that may be employed in the training of the classification engine.

FIG. 4A shows training healthy manifolds learned from a trained auto-encoder as an example of a severity analysis module.

FIG. 4B shows representative examples of OCT scans with high and low severity scores employed in the study.

FIG. 4C shows an example training operation using an auto-encoder for severity score analysis.

FIG. 4D shows a flowchart for a supervised contrastive learning operation performed using severity labels.

FIG. 5A shows the performance of severity labeling and supervised contrastive learning approach on individual biomarkers. Multi-Label is the average AUC from the multi-label classification task. S_{LN} refers to training an encoder after having divided the training set into N severity label bins.

FIG. 5B shows the comparative performance of different anomaly detectors to generate severity labels.

### DETAILED SPECIFICATION

To facilitate an understanding of the principles and features of various embodiments of the present invention, they are explained hereinafter with reference to their implementation in illustrative embodiments.

### Example System

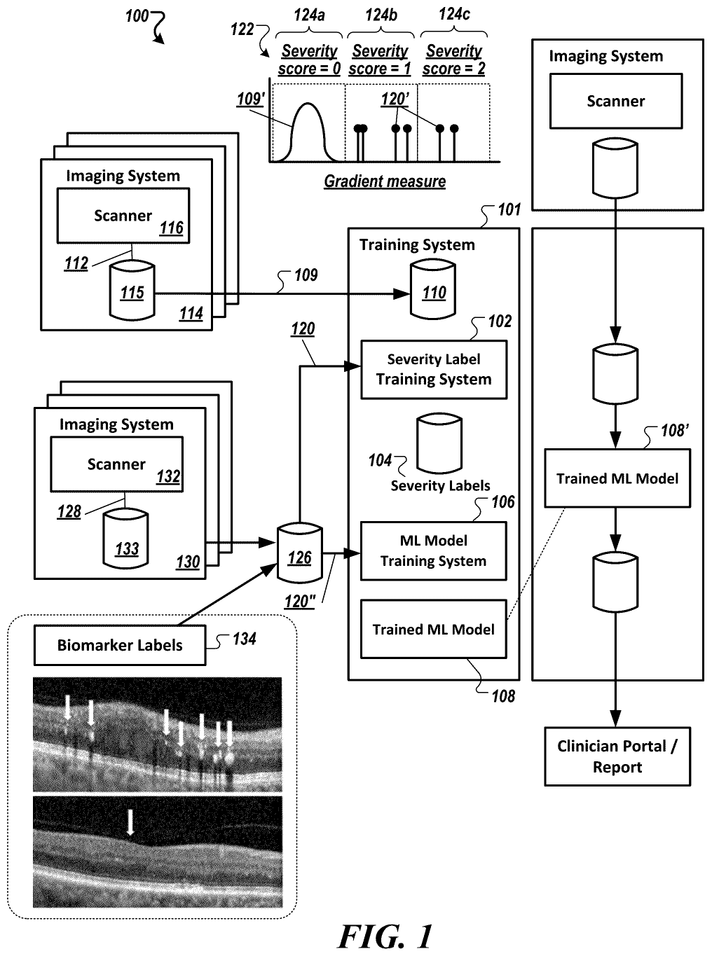

FIG. 1 shows an example system 100 configured with severity analysis module 102 (shown as “Severity Label Training System” 102) to perform contrastive learning to generate pseudo-severity-based labels 104 for unlabeled medical images to train, via a training module 106 (shown as “ML Model Training System” 106), a classification engine 108 (shown as “trained ML model” 108) in accordance with an illustrative embodiment. The classification engine 108 can then be used for diagnostics or treatment of a disease or medical condition.

In the example shown in FIG. 1, the severity analysis module 102 is configured to receive a contrastive-learning training data set 109 from a data store 110. The data store 110 may be located on an edge device, a server, or cloud infrastructure to receive the scanned medical images 112 from an imaging system 114 comprising a scanner 116. The imaging system 114 can acquire scans for optical coherence tomography, ultrasound, magnetic resonance imaging, and computing tomography, among other modalities described or referenced herein. The scanned data can be stored in a local data store 115 to then be provided as the training data set 109 to the training system 101.

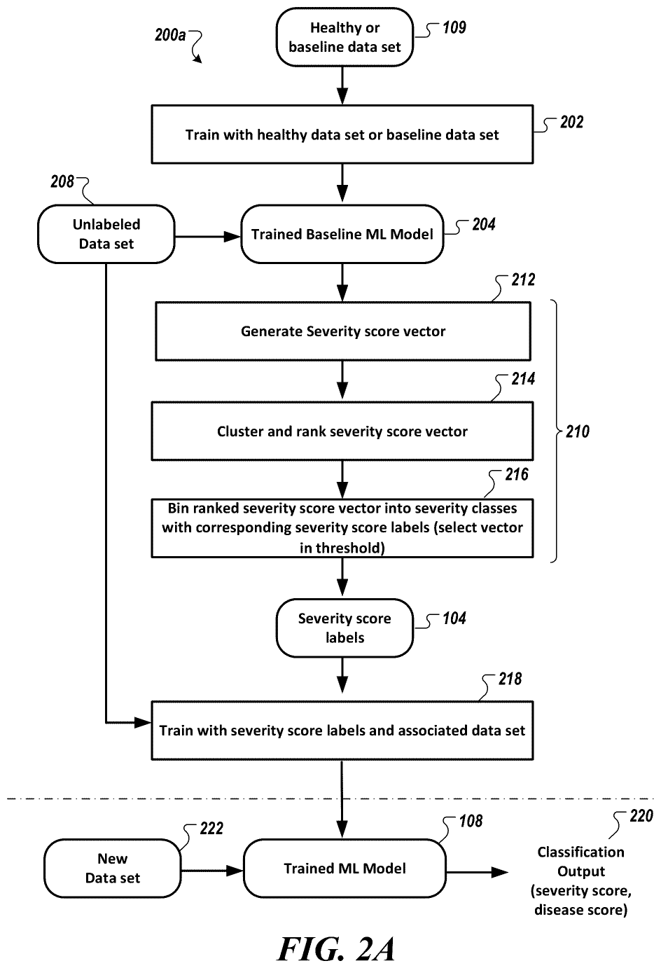

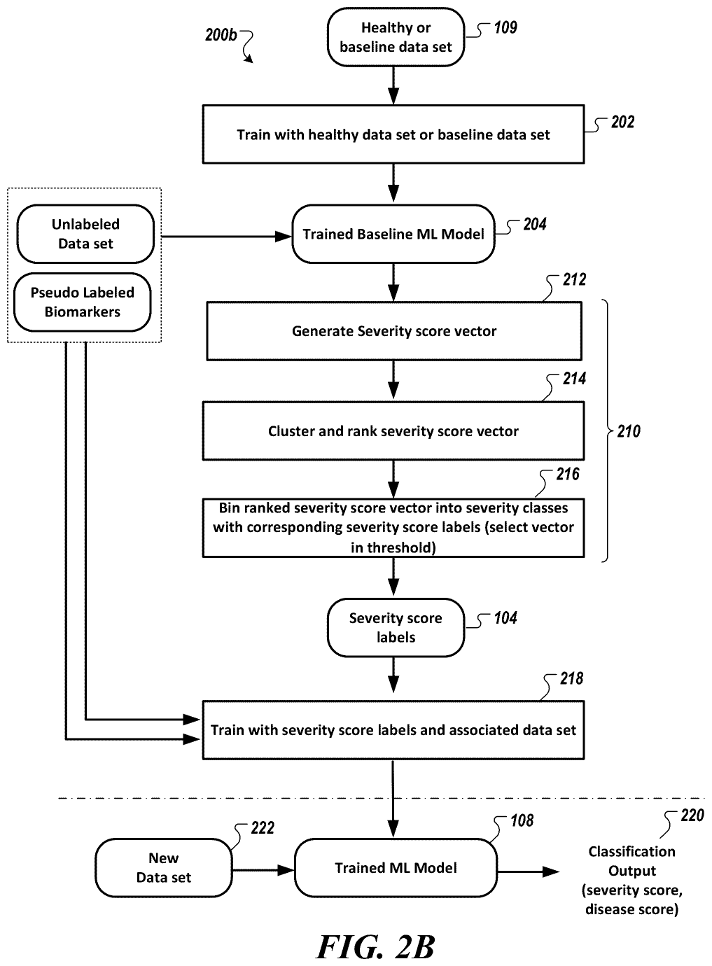

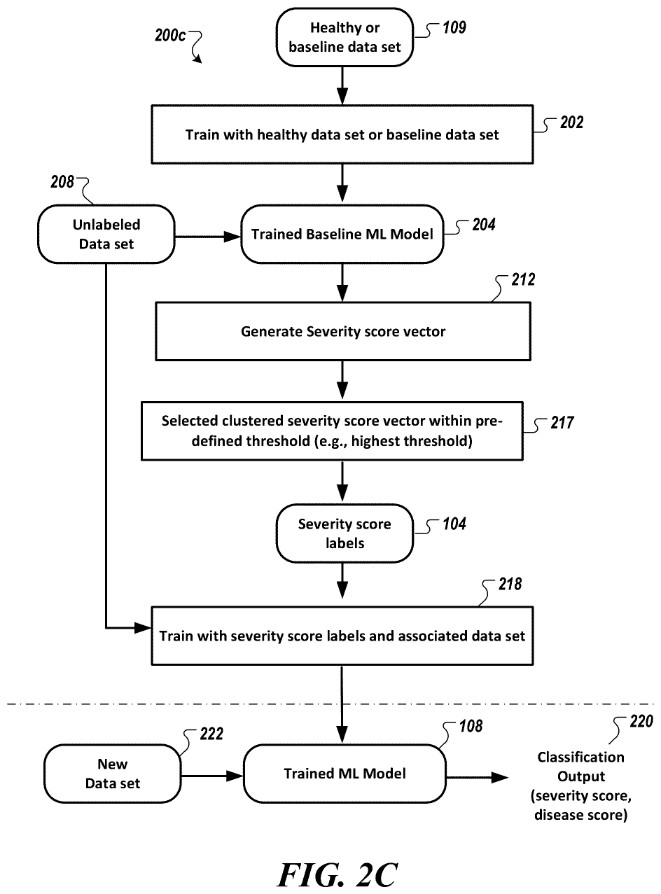

The contrastive-learning training data set 109, as illustratively shown in diagram 122 of the operation, employs healthy or baseline data that are first used to train a model (e.g., 204, see FIG. 2A) (e.g., autoencoder or other ML models described herein) of the severity analysis module 102 in which the model can be used to establish a baseline (shown as 109′) for gradient measures for a subsequent anomaly detection-based operation with respect to an unlabeled data set 120 (shown as 120′). As shown in diagram 122, the gradient measures of the healthy/baseline data set 109′ and subsequent data set 120′ are then clustered or grouped 124 (shown as a “severity score=0” group 124a, a “severity score=1” group 124b, and a “severity score=2” group 124c). The clusters or groups are then assigned severity labels 104 that can then be used for diagnosis of a disease or medical condition or as labels for as a training data set for training a classification model of the same. In the example shown in FIG. 1, once the pseudo-severity-based labels 104 have been generated for unlabeled medical images, they can be employed in a training operation, via the training module 106, as a second set of training data for the classification engine 108. Severity labels can be generated, for example, any degree of carcinomas, sarcomas, lymphomas, leukemias, germ cell tumors, blastomas, or other diseases or conditions described herein.

The clustering or grouping can be performed by a clustering operation followed by a ranking/sorting operation of a severity score vector generated by the trained model (e.g., auto-encoder, etc.) of the severity analysis module 102. In other embodiments, the clustering or grouping can be performed by selecting a portion of the scores within the severity score vector (e.g., highest or lowest by a defined threshold).

The severity analysis module 102 is configured to receive unlabeled data set 120 from a data store 126. The data store 126 may be located on an edge device, a server, or cloud infrastructure to receive the scanned medical images 128 from an imaging system 130 comprising a scanner 132. The imaging system 130 can acquire scans for optical coherence tomography, ultrasound, magnetic resonance imaging, and computing tomography, among other modalities described or referenced herein. The scanned data can be stored in a local data store 133 to then be provided as the training data set 120 (shown as 120″) to the training system 106 along with the corresponding severity labels 104.

The training performed at the ML model training system 106 can be performed in a number of different ways. The ML model training system 106 can be employed to use all the generated severity labels 104, and corresponding data set 120″ for the training, in which the generated severity labels 104 is employed as ground truth. The resulting classification engine 108 (shown as 108′) can then be used to generate an estimated/predicted severity label/score for a new data set in a clinical application. In such embodiments, the classification engine 108′ can additionally generate both an indication for the presence or non-presence of a disease or medical condition as a corresponding severity score for the disease or condition.

In a second embodiment, the ML model training system 106 can be employed to use some of the generated severity labels 104 (e.g., severest or higher tier severe scores) and corresponding data set 120″ for the training, in which the selected severity labels 104 is employed as ground truth. The resulting classification engine 108 (shown as 108′) can then be used to generate (i) an estimated/predicted severity label/score for a new data set in a clinical application or (ii) a presence of an indication of a disease or medical condition. In some embodiments, the resulting classification engine 108′ can then be used to generate an indication of a presence of non-disease or condition (i.e., healthy indication).

Referring still to FIG. 1, the output of the classification engine 108′ can be outputted via a report or display, e.g., for the diagnosis of a disease or a medical condition and/or for the treatment of the disease or a medical condition. Treatment refers to operations of medical instruments that operate on a tissue, e.g., to excise, remove, cut, ablate, or cool a tissue. Treatment can also refer to the introduction (e.g., injection) of a therapeutic agent. In some embodiments, an edge device, server, or cloud infrastructure can employed, e.g., via web services, to curate a clinician or healthcare portal to display the report or information in a graphical user interface.

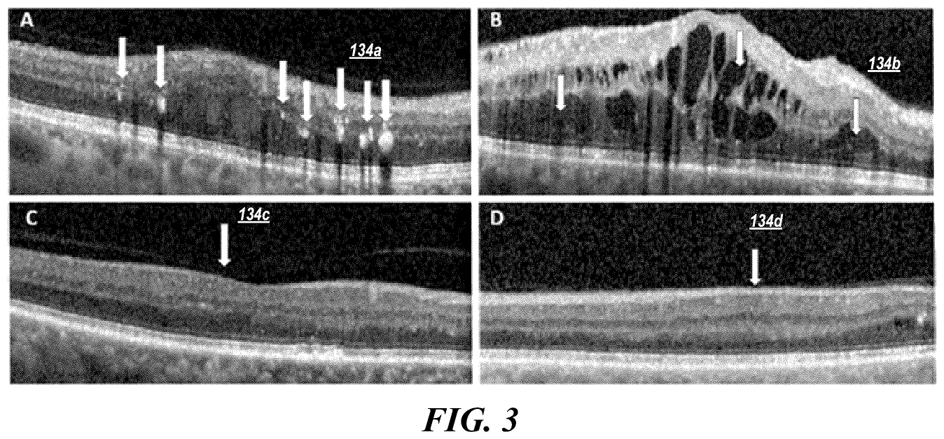

Biomarker training. The training system 106 can train the severity labels 104 and associated training dataset 120″, which can be marked with biomarker data. Biomarkers can include any substance, structure, or process that can be measured in the body or its products and influence or predict the incidence of outcome or disease. In the context of Diabetic Retinopathy, biomarkers can include, for example, but not limited to, the presence or degree of Intraretinal Fluid (IRF), Diabetic Macular Edema (DME), Intra-Retinal Hyper-Reflective Foci (IRHRF), atrophy or thinning of retinal layers, disruption of the ellipsoid zone (EZ), disruption of the retinal inner layers (DRIL), intraretinal (IR) hemorrhages, partially attached vitreous face (PAVF), fully attached vitreous face (FAVF), preretinal tissue or hemorrhage, vitreous debris, vitreomacular traction (VMT), diffuse retinal thickening or macular edema (DRT/ME), subretinal fluid (SRF), disruption of the retinal pigment epithelium (RPE), serous pigment epithelial detachment (PED), subretinal hyperreflective material (SHRM). FIG. 1 (and FIG. 3) shows, for OCT scans (from Prime and TREX DME datasets), biomarkers that include Intraretinal Hyperreflective Foci (IRHRF) (134a), Intraretinal Fluid (IRF) and Diabetic Macular Edema (DME) (134b), Partially Attached Vitreous Face (PAVF) (134c), and Fully Attached Vitreous Face (FAVF) (134d). Additional examples of biomarkers in OCT can be found at [2].

In addition to images, the example system of FIG. 1 can be employed to evaluate other image and sensor data, e.g., optical, temperature, acoustic, sound, strain/stress, etc, as employed in clinical, engineering, and metrology applications. The exemplary system, for example, can be employed for Clinical Disease Detection, Clinical diagnosis analysis, X-ray interpretation, OCT interpretation, Ultrasound Interpretation, Infrastructure Assessment, Structure Integrity assessment, Industrial applications, Manufacturing applications, and Circuit Boards defect detection systems, among others.

### Example Training Operation

FIGS. 2A, 2B, 2C each shows an example method 200 (shown as 200a, 200b, 200c, respectively) to perform contrastive learning to generate pseudo-severity-based labels (e.g., 104) for unlabeled medical images to train a classification engine (e.g., 108) in accordance with an illustrative embodiment. Method (200a, 200b, or 200c) includes in a contrastive learning operation, training (202) a baseline ML model 204 via a first data set 109 that includes only data for a non-anomalous, normal, or healthy set (e.g., patient, sample, etc.).

Method (e.g., 200a, 200b, 200c) then includes generating (206) gradient severity score vector from the baseline ML model 204 for a second data set (e.g., 120″, now shown as 208) that includes anomalous or unhealthy set as well as healthy data. The second data set 208 was provided as unlabeled with respect to severity.

Method (e.g., 200a, 200b) then includes tiering (210) the severity score vector into a plurality of severity classes by clustering (212) the severity scores of the second data set into a vector and then ranking/sorting (214) the scores. The ranked/sorted scores are then binned (216) into severity classes to which severity labels (104) can be applied. The severity scores are generated based on the properties of the network used to train the healthy data. Example statistics or parameters that capture the severity of samples as an anomaly from the healthy/baseline data set include the reconstruction error, gradient response induced by a sample, and 11-norm of a latent space vector.

Method 200c shows an alternative approach. In FIG. 2C, Method 200c includes selecting a portion of the severity scores in a pre-defined threshold or range, e.g., to select the highest or lowest portions or groups of the severity scores 211.

Method (e.g., 200a, 200b, 200c) can employ all the generated severity labels 104 and corresponding data set 208 in a training operation (218), in which the generated severity labels 104 is employed as ground truth. The resulting classification engine 108 can then be used to generate an estimated/predicted severity label/score 220 for a new data set 222 in a clinical application. In such embodiments, the classification engine 108 (shown as an example of a “Trained ML Model”) can additionally generate both an indication for a presence or non-presence of a disease or medical condition as a corresponding severity score for the disease or condition.

In another embodiment, Method 200a, 200b, 200c can employ some of the generated severity labels 104 (e.g., severest or higher tier severe scores) and corresponding data set 208 for the training, in which the selected severity label 104 is employed as ground truth. The resulting classification engine 108 can then be used to generate (i) an estimated/predicted severity label/score for a new data set in a clinical application or (ii) a presence of indication of a disease or medical condition. In some embodiments, the resulting classification engine 108 can then be used to generate an indication of the presence of a non-disease or condition (i.e., healthy indication).

In FIG. 2A, Method 200a employs only the unlabeled data set in the training and severity score analysis. In FIG. 2B, Method 200b employs both the unlabeled data set in the training and severity score analysis along with biomarkers labeled within the image data. The biomarkers, via the training, can be associated with a severity score.

The classification engine 108, e.g., as described in relation to FIGS. 1, 2A, 2B, 2C, as well as the trained ML model of the severity score analysis module, can be implemented using one or more artificial intelligence and machine learning operations. The term “artificial intelligence” can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as a feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as an input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., an error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, and depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by down sampling). A fully-connected layer includes neurons, where each neuron is connected to all of the neurons in the previous layer. The layers are stacked similar to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., error such as L1 or L2 loss), during training. This disclosure contemplates that any algorithm that finds the minimum of the cost function can be used. LR classifiers are known in the art and are therefore not described in further detail herein.

A Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

### Experimental Results and Additional Examples

A study was conducted to develop contrastive learning operations that can generate pseudo-severity-based labels for unlabeled optical coherence tomography (OCT) medical images. In the study, natural images were analyzed using contrastive learning for data augmentation by selecting positive and negative pairs for contrastive loss. In the medical domain, arbitrary augmentations can distort small, localized regions that contain biomarkers of interest. Samples with similar disease severity characteristics can have similar structures to that of the progression of the disease. The study can generate disease severity labels for unlabeled OCT scans on the basis of gradient responses from an anomaly detection algorithm. The labels are used to train a supervised contrastive learning setup to improve biomarker classification accuracy by as much as 6% above supervised and self-supervised baselines for key indicators of Diabetic Retinopathy.

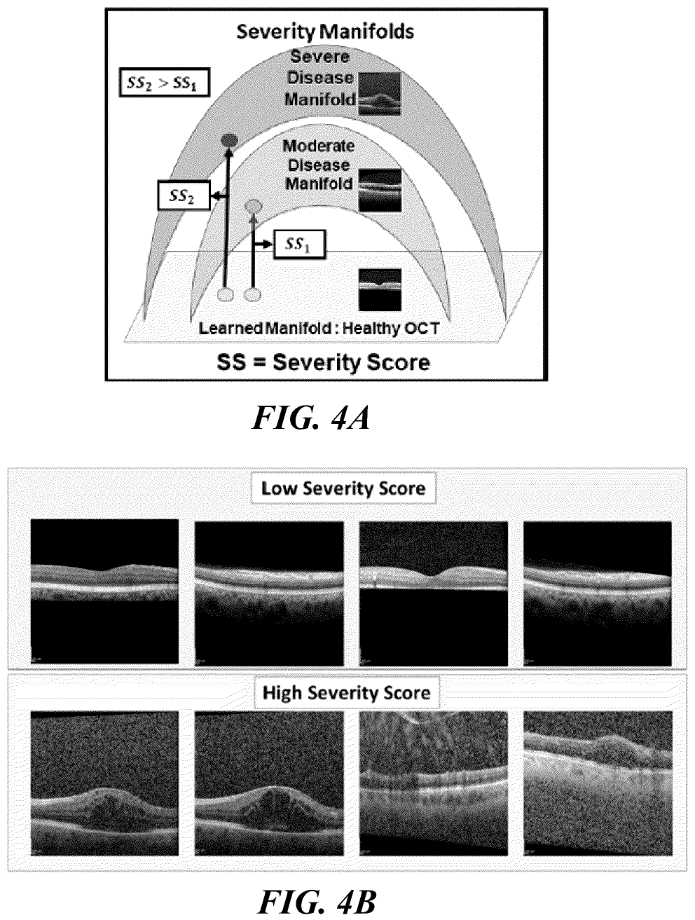

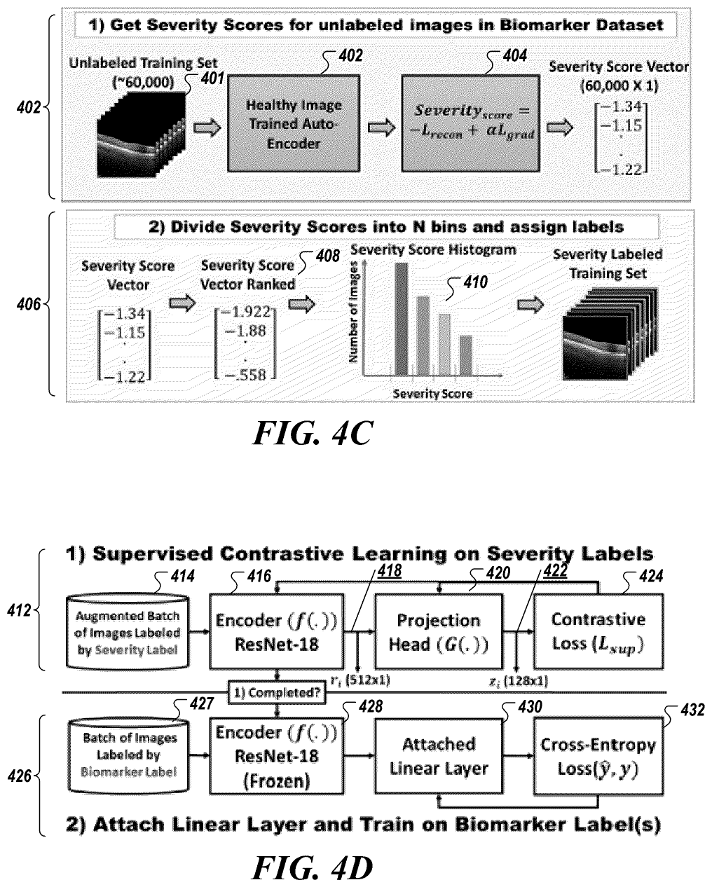

FIG. 4A shows training healthy manifolds learned from a trained auto-encoder. The computed distance of the manifold of more severely diseased cases can be used as a Severity Score (SS). Severity Score was calculated via a model response and increased as a sample is more anomalous compared to the learned healthy manifold.

Training Data Set and Training Methodology. FIG. 4C shows an example training operation using an auto-encoder. In the study, two datasets of interest were used, including healthy images and a Biomarker dataset from the Kermany dataset [23] and Prime+TREX DME [24,25]. The training set of the Biomarker dataset included approximately 60,000 unlabeled OCT scans and 7,500 OCT scans (401) with explicit biomarker labels from 76 different eyes. The OCT scans were collected as part of the Prime and TREX DME studies at the Retina Consultants of Texas (RCTX). For each labeled OCT scan in the Biomarker dataset, a trained grader performed interpretation on OCT scans for the presence or absence of 20 different biomarkers. Among the biomarkers, 5 existed in balanced quantities to be used in training for the binary classification task of detecting the biomarker of interest. OCT scans from 76 eyes constituted the training set, and the OCT scans from a remaining set of 20 eyes make up the test set. From the test OCT scans, random sampling was employed to develop an individualized test set for each of the 5 biomarkers used in our analysis. The analysis was performed for each biomarker in a balanced test set with 500 OCT scans with the biomarker present and 500 OCT Scans with the biomarker absent was generated. FIG. 4B shows representative examples of OCT scans with high and low severity scores employed in the study.

In the study, and as shown in FIG. 4C, the study first evaluated the distribution of healthy OCT scans. To do so, the study preprocessed the data by resizing healthy images from the Healthy dataset to 224×224 pixels and trained (402) an auto-encoder through the GradCON methodology [13]. The training introduced a gradient constraint so that gradients from healthy images will align more closely together. The study causes images that deviate from the healthy distribution to have gradients that are more distinguishable.

As shown in diagram 402, the study determined (404) the severity score for all unlabeled images in the Biomarker dataset. To do this, the example embodiment can pass all unlabeled images to the input of the trained auto-encoder network and extract their corresponding severity score as a vector per Equation 1.

Severity_{Score}=−L_{recon}+αL_{grad} (Eq. 1)

In Equation 1, L_{recon} is the mean squared error between an input x and its reconstructed output \hat{x}, L_{grad} is the average of the cosine similarity between the gradients of the target image, and the reference gradients learned from training on the healthy dataset across all layers of every image having an associated severity score which constitutes a severity score vector.

Referring still to FIG. 4C, the study then generated (406) severity labels for each image are generated. In the study, the study ranked (408) the determined severity scores in the vector in ascending order and then divided (410) the ranked scores into N bins based on similarity of the severity. Images belonging to the same severity score bin are assigned the same severity label (SL) for subsequent analyses. The previously unlabeled data now have one of N possible labels. N is a hyperparameter that is explored experimentally. To get an intuitive sense of the quality of these severity labels, the study sampled the images randomly from the bins at the extreme ends of the histogram. FIG. 4B shows a visual representation of OCT scans with high and low severity scores. In FIG. 4B, it can be observed that the lower severity scores correspond with images that are healthier than those at the other end of the distribution. This makes sense as lower severity scores indicate that the image's gradients have a greater alignment with the gradients of the learned healthy distribution.

FIG. 4D shows a flowchart for a supervised contrastive learning operation performed using severity labels. The supervised contrastive loss [14] was applied to the severity labels (SL) to bring embeddings of images with the same label together and push apart embeddings of images with differing labels. In FIG. 4D, the supervised contrastive learning was performed along with linear fine-tuning steps. The supervised contrastive loss was performed on the generated severity labels from previously unlabeled data. The second operation in FIG. 4D entailed training an attached linear layer on labeled biomarker data.

In FIG. 4D, the first step 412 involves training (412) an encoder with the supervised contrastive loss. Each image xi (414) is passed (416) through an encoder network f(⋅), a ResNet-18 [26], to produce a 512×1-dimensional vector ri (418). The vector is then compressed through a projection head G(⋅) (420) configured with a multi-layer perceptron with a single hidden layer. The projection head 420 reduced the dimensionality of the representation and was discarded after training. The output (422) of G(⋅) is a 128×1 dimensional embedding zᵢ. In this embedding space, the dot product of images with the same severity label (the positive samples) were maximized, and those with different severity labels (the negative samples) were minimized, shown as contrastive loss Lₛᵤₚ (424), per Equation 2. In Equation 2, positive instances for image xᵢ come from the set P(i), and positive and negative instances come from the set A(i). τ is a temperature scaling parameter, which was set to 0.07 for all experiments.

$${L}_{sup} = {\sum }_{i ∈ I} \frac{- 1}{\vert P ( i ) \vert} {\sum }_{p ∈ P ( i )} log \frac{exp ( {𝓏}_{i} \cdot {𝓏}_{p} / τ )}{{\sum }_{a ∈ A ( i )} exp ( {𝓏}_{i} \cdot {𝓏}_{a} / τ )} \qquad ( Eq . 2 )$$

At step 2 (426), the model is configured to explicitly learn to detect biomarkers, utilizing a subset of the Biomarker dataset (427) with biomarker labels for fine-tuning on top of the representation space learned in step 1 (412). To do this, the study froze (428) the weights of the encoder and appended (430) a linear layer to the output of the encoder. The training chooses a biomarker to be a biomarker of interest, and the linear layer is trained (432) using cross-entropy loss between a predicted output ŷ and a ground truth label y to learn to detect the presence or absence of the biomarker in the image.

Results. The study compared the instant training against a fully supervised setup using a cross-entropy loss on the Biomarker dataset with biomarker labels as well as three state-of-the-art self-supervised strategies that make use of the unlabeled data. The architecture was kept constant as ResNet-18 across all experiments. Augmentations included random resize crop to a size of 224, horizontal flip, color jitter, and normalization to the mean and standard deviation of the respective dataset. The batch size was set at 64. Training was performed for 25 epochs in every setting. A stochastic gradient descent optimizer was used with a learning rate of 1e-3 and a momentum of 0.9. The accuracy and F1-score were recorded for the testing of performance on cach individual biomarker.

Additionally, the study assessed the exemplary method's capability across all biomarkers by utilizing a mean AUC metric within a multi-label classification setting for the labels of all 5 biomarkers at the same time. Overall, intelligent choosing of the severity bin hyperparameter N leads to performance improvements in both multi-label classification performance as well as detection performance on individual biomarkers.

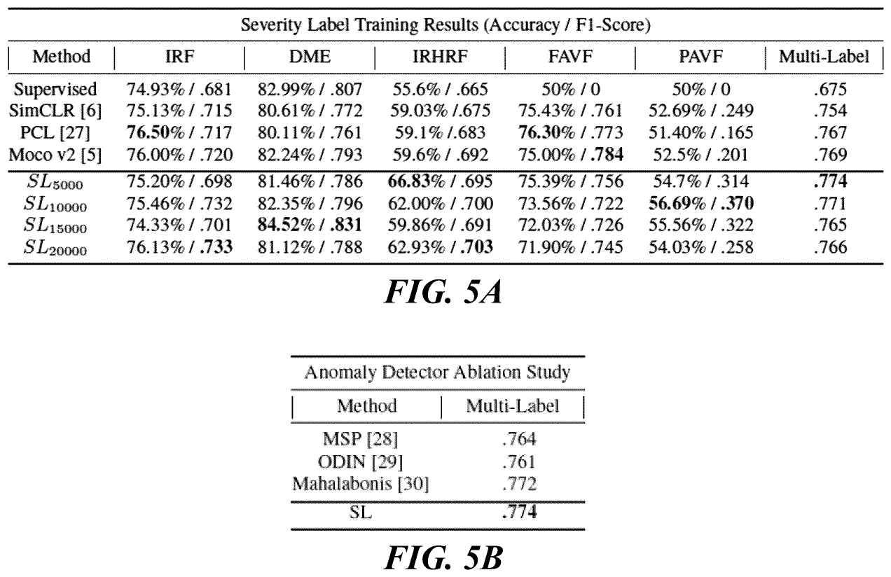

FIG. 5A shows a table with a summary of the results of the study. SLN represents the encoder trained after dividing the unlabeled pool into N severity label bins. This is repeated for different numbers of severity bin divisions ranging from 5000 to 20000. A larger number of bins means the images within each bin are more likely to share structures in common but at the trade-off of having fewer positive instances during training. A more moderate choice for a number of severity bins, such as 5000 or 10000, led to improved performance in multi-class classification compared to all baselines. As shown in FIG. 5A, it was observed that choosing larger or smaller values for the number of bins resulted in the best performance for specific biomarkers. For example, the best-performing values for DME and IRF resulted in the higher number of severity bins of 15000 and 20000. Additionally, the best result for PAVF was at a severity bin value of 10000. The variation in performance for different bin numbers can also be understood from the perspective of how fine-grained individual biomarkers are. IRF and DME manifest themselves more distinctively than IRHRF, FAVF, or PAVF. Therefore, it's possible that a lower number of more closely related positives may be better for identifying these distinctive features. However, in the case of more difficult-to-identify biomarkers, it may be the case that some level of diversity in the positives is necessary to identify them from the surrounding structures in the OCT scan.

The study also evaluated the effect of using other anomaly detection methods to generate severity scores. The study trained a classifier using the labeled data from the Biomarker dataset. Using the output logits from this classifier, the study generated anomaly scores for each of the methods shown in FIG. 5B. The scores were processed, as described in relation to FIG. 4C, to generate labels for the unlabeled dataset. Specifically, FIG. 5B shows the comparative performance of different anomaly detectors to generate severity labels.

The labels are used, as described in relation to FIG. 4D, to train an encoder with a supervised contrastive loss. The study measured performance as the average AUC value from a multi-label classification setting. It can be observed that the instant method for the same level of discretization outperforms all other anomaly detection methodologies.

### Discussion

Current technologies rely on the classification of an explicit label. A problem with this is that obtaining a large dataset of labeled severity is intractable. This is partially because it is expensive to obtain experts to perform this interpretation, but the problem extends to the fact that severity exists on a continuous distribution. Therefore, any assigned label will not truly be able to reflect the severity properties of the image. By having a method that can estimate severity directly, embodiments of the present disclosure can overcome both of these challenges.

Diabetic Retinopathy (DR) is the leading cause of irreversible blindness among people aged 20 to 74 years old [1]. In order to manage and treat DR, the detection and evaluation of biomarkers of the disease is a necessary step for any clinical practice [2]. Biomarkers can refer to any substance, structure, or process that can be measured in the body or its products and influence or predict the incidence of outcome or disease [3]. Biomarkers such as those in FIG. 1 are important indicators of DR and give ophthalmologists a fine-grained understanding of the manifestation of DR for individual patients.

Due to the importance of biomarkers in the clinical decision-making process, much work has gone into deep learning methods to automate their detection directly from optical coherence tomography (OCT) scans [4]. A major bottleneck hindering this goal is the dependence of conventional deep learning architectures on having access to a large training pool. This dependency is not generalizable to the medical domain, where biomarker labels are expensive to curate due to the requirement of expert graders. In order to move beyond this limitation, contrastive learning [5] has been one of the research directions to leverage the larger set of unlabeled data to improve performance on the smaller set of labeled data. Contrastive learning approaches operate by creating a representation space by minimizing the distance between positive pairs of images and maximizing the distance between negative pairs. Traditional approaches like [6] generate positive pairs by taking augmentations from a single image and treating all other images in the batch as the negative pairs. While such operation may be beneficial from a natural image perspective, from a medical point of view, the augmentations utilized in these strategies, such as Gaussian blurring, can potentially distort small localized regions that contain the biomarkers of interest. Examples of regions that could potentially be occluded are indicated by white arrows in FIG. 3.

The exemplary system and method employ a more intuitive approach from a medical perspective by selecting positive pairs that are at a similar level of severity. Images with similar disease severity levels share structural features in common that manifest themselves during the progression of DR [7]. Hence, choosing positive pairs on the basis of severity can better bring together OCT scans with similar structural components in contrastive loss. It is also possible to view more severe samples as existing on a separate manifold from the healthy trained images as shown in FIG. 4A.

From this manifold outlook of severity, model responses can be calculated as a severity score that indicates how far a sample is from the healthy manifold. To capture this intuition, “severity” is described herein as “the degree to which a sample appears anomalous relative to the distribution of healthy images.” From this perspective, one way to measure severity is by formulating it as an anomaly detection problem where some response from a trained network can serve to identify the degree to which a sample differs from healthy images through a severity score.

Embodiments of the present disclosure can measure the gradient from the update of a model. Gradients represent the model update required to incorporate new data. From this intuition, gradients have been shown to be able to represent the learned representation space from a model [8], represent contrastive explanations between classes [9], and perform contrastive reasoning [10]. Anomalous samples require a more drastic update to be represented than normal samples [11]. Additionally, previous work [12] showed that gradient information could be used to effectively rank samples into subsets that exhibit semantic similarities. Hence, embodiments of the present disclosure can use gradient measures from an anomaly detection methodology known as GradCON [13] to assign pseudo severity labels to a large set of unlabeled OCT scans. The example embodiment utilizes these severity labels to train an encoder with a supervised contrastive loss [14] and then fine-tune the representations learned on a smaller set of available biomarker labels. In this way, the example embodiment can leverage a larger set of readily obtainable healthy images and unlabeled data to improve performance in a biomarker classification task.

While the present disclosure has been described with reference to certain types of medical images as a non-limiting example, it should be understood that embodiments described herein can be used in any setting where interpretation of the disease characteristics of medical data is necessary. This can include aiding in analysis of radiologists to helping diagnostic decisions by routine-care practitioners.

Additional non-limiting example applications include: Clinical Disease Detection, Clinical diagnosis analysis, X-ray interpretation, OCT interpretation, Ultrasound Interpretation, Infrastructure Assessment, Structure Integrity assessment, Industrial applications, Manufacturing applications, and Circuit Boards defect detection systems.

Various sizes and dimensions provided herein are merely examples. Other dimensions may be employed.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, clement, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

It should be appreciated that the logical operations described above and, in the appendix, can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The following patents, applications and publications as listed below and throughout this document are hereby incorporated by reference in their entirety herein.
[1] Donald S Fong. Lloyd Aiello, Thomas W Gardner, George L King, George Blankenship, Jerry D Cavallerano, Fredrick L Ferris III, Ronald Klein, and American Diabetes Association, “Retinopathy in diabetes,” Diabetes care, vol. 27, no. suppl 1, pp. s84-s87. 2004.
[2] Ashish Markan, Aniruddha Agarwal, Atul Arora, Krinjeela Bazgain, Vipin Rana, and Vishali Gupta, “Novel imaging biomarkers in diabetic retinopathy and diabetic macular edema,” Therapeutic Advances in Ophthalmology, vol. 12, pp. 2515841420950513, 2020.
[3] Kyle Strimbu and Jorge A Tavel, “What are biomarkers?,” Current Opinion in HIV and AIDS, vol. 5, no. 6, pp. 463, 2010.
[4] Daniel S Kermany, Michael Goldbaum, Wenjia Cai, Carolina CS Valentim, Huiying Liang, Sally L Baxter, Alex Mckeown, Ge Yang, Xiaokang Wu, Fangbing Yan, et al., “Identifying medical diagnoses and treatable diseases by image-based deep learning.” Cell, vol. 172, no. 5, pp. 1122-1131, 2018.
[5] Xinlei Chen, Haoqi Fan, Ross Girshick, and Kaiming He, “Improved baselines with momentum contrastive learning.” arXiv preprint arXiv:2003.04297, 2020.
[6] Ting Chen, Simon Kornblith, Mohammad Norouzi, and Geoffrey Hinton, “A simple framework for contrastive learning of visual representations,” in International conference on machine learning. PMLR, 2020, pp. 1597-1607.
[7] Xiaogang Wang, Yongqing Han, Gang Sun, Fang Yang, Wen Liu, Jing Luo, Xing Cao, Pengyi Yin, Frank L Myers, and Liang Zhou, “Detection of the microvascular changes of diabetic retinopathy progression using optical coherence tomography angiography.” Translational Vision Science & Technology, vol. 10, no. 7, pp. 31-31, 2021.
[8] Gukyeong Kwon, Mohit Prabhushankar, Dogancan Temel, and Ghassan AlRegib, “Distorted representation space characterization through backpropagated gradients,” in 2019 IEEE International Conference on Image Processing (ICIP). IEEE, 2019, pp. 2651-2655.
[9] Mohit Prabhushankar, Gukyeong Kwon, Dogancan Temel, and Ghassan AlRegib, “Contrastive explanations in neural networks,” in 2020 IEEE International Conference on Image Processing (ICIP). IEEE, 2020, pp. 3289-3293.
[10] Mohit Prabhushankar and Ghassan AlRegib, “Contrastive reasoning in neural networks,” arXiv preprint arXiv:2103.12329, 2021.
[11] Jinsol Lee and Ghassan AlRegib, “Gradients as a measure of uncertainty in neural networks.” in 2020 IEEE International Conference on Image Processing (ICIP). IEEE, 2020, pp. 2416-2420.
[12] Chirag Agarwal, Daniel D'souza, and Sara Hooker, “Estimating example difficulty using variance of gradients,” arXiv preprint arXiv:2008.11600, 2020.
[13] Gukyeong Kwon, Mohit Prabhushankar, Dogancan Temel, and Ghassan AlRegib, “Backpropagated gradient representations for anomaly detection,” in European Conference on Computer Vision. Springer, 2020, pp. 206-226.
[14] Prannay Khosla, Piotr Teterwak, Chen Wang, Aaron Sarna, Yonglong Tian, Phillip Isola, Aaron Maschinot, Ce Liu, and Dilip Krishnan, “Supervised contrastive learning.” arXiv preprint arXiv:2004.11362, 2020.
[15] Dogancan Temel, Melvin J Mathew, Ghassan AlRegib, and Yousuf M Khalifa, “Relative afferent pupillary defect screening through transfer learning.” IEEE Journal of Biomedical and Health Informatics, vol. 24, no. 3, pp. 788-795, 2019.
[16] Antoine Rivail, Ursula Schmidt-Erfurth, Wolf-Dieter Vogl, Sebastian M Waldstein, Sophie Riedl, Christoph Grechenig, Zhichao Wu, and Hrvoje Bogunovic, “Modeling disease progression in retinal octs with longitudinal self-supervised learning.” in International Workshop on PRedictive Intelligence In MEdicine. Springer, 2019, pp. 44-52.
[17] Yuexiang Li, Jiawei Chen, Xinpeng Xie, Kai Ma, and Yefeng Zheng, “Self-loop uncertainty: A novel pseudo-label for semisupervised medical image segmentation,” in International Conference on Medical Image Computing and Computer-Assisted Intervention. Springer, 2020, pp. 614-623.
[18] Yuhan Zhang, Mingchao Li, Zexuan Ji, Wen Fan, Songtao Yuan, Qinghuai Liu, and Qiang Chen, “Twin self-supervision based semi-supervised learning (ts-ssl): Retinal anomaly classification in sd-oct images,” Neurocomputing, 2021.
[19] Hari Sowrirajan, Jingbo Yang, Andrew Y Ng, and Pranav Rajpurkar, “Moco-cxr: Moco pretraining improves representation and transferability of chest x-ray models,” arXiv preprint arXiv:2010.05352. 2020.
[20] Yen Nhi Truong Vu, Richard Wang, Niranjan Balachandar, Can Liu, Andrew Y Ng, and Pranav Rajpurkar, “Medaug: Contrastive learning leveraging patient metadata improves representations for chest x-ray interpretation,” arXiv preprint arXiv:2102.10663, 2021.
[21] Joseph Y Cheng, Hanlin Goh, Kaan Dogrusoz, Oncel Tuzel, and Erdrin Azemi, “Subject-aware contrastive learning for biosignals,” arXiv preprint arXiv:2007.04871, 2020.
[22] Dewen Zeng, Yawen Wu, Xinrong Hu, Xiaowei Xu, Haiyun Yuan, Meiping Huang, Jian Zhuang, Jingtong Hu, and Yiyu Shi, “Positional contrastive learning for volumetric medical image segmentation,” arXiv preprint arXiv:2106.09157, 2021.
[23] Daniel Kermany, Kang Zhang, and Michael Goldbaum, “Large dataset of labeled optical coherence tomography (oct) and chest x-ray images,” Mendeley Data, vol. 3, pp. 10-17632, 2018.
[24] John F Payne, Charles C Wykoff, W Lloyd Clark, Beau B Bruce, David S Boyer, and David M Brown, “Long-term outcomes of treat-and-extend ranibizumab with and without navigated laser for diabetic macular oedema: Trex-dme 3-year results,” British Journal of Ophthalmology, vol. 105, no. 2, pp. 253-257, 2021.
[25] J Yu Hannah, Justis P Ehlers, Duriye Damla Sevgi, Jenna Hach, Margaret O'Connell, Jamie L Reese, Sunil K Srivastava, and Charles C Wykoff, “Real-time photographic-and fluorescein angiographic-guided management of diabetic retinopathy: Randomized prime trial outcomes,” American Journal of Ophthalmology, vol. 226, pp. 126-136, 2021.
[26] Kaiming He, Xiangyu Zhang, Shaoqing Ren, and Jian Sun, “Deep residual learning for image recognition,” in Proceedings of the IEEE conference on computer vision and pattern recognition, 2016, pp. 770-778.
[27] Junnan Li, Pan Zhou, Caiming Xiong, and Steven CH Hoi, “Prototypical contrastive learning of unsupervised representations,” arXiv preprint arXiv:2005.04966, 2020.
[28] Dan Hendrycks and Kevin Gimpel, “A baseline for detecting misclassified and out-of-distribution examples in neural networks,” arXiv preprint arXiv: 1610.02136, 2016.
[29] Shiyu Liang, Yixuan Li, and Rayadurgam Srikant, “Enhancing the reliability of out-of-distribution image detection in neural networks,” arXiv preprint arXiv:1706.02690, 2017.
[30] Kimin Lee, Kibok Lee, Honglak Lee, and Jinwoo Shin, “A simple unified framework for detecting out-of-distribution samples and adversarial attacks,” Advances in neural information processing systems, vol. 31, 2018.

What is claimed is:

1. A method of training a machine learning model, the method comprising:
in a contrastive learning operation, training a baseline ML model via a first data set, the first data set consisting of data for a non-anomalous, normal, or healthy set;
in the contrastive learning operation, generating gradient severity score vector from the baseline ML model for a second data set, the second data set comprising data for anomalous or unhealthy set, wherein the second data set is unlabeled with respect to severity; and
in the contrastive learning operation, tiering the severity score vector into a plurality of severity classes, including a first severity class associated with a first severity score label and a second severity class associated with a second severity score label;
wherein at least one of the first severity score label and the second severity score label is used (i) for diagnosis or (ii) as labels for the second data set as a training data set for a second ML model or the baseline ML model.

2. The method of claim 1, wherein the step of tiering the severity score vector into the plurality of severity classes comprises:
ordering the severity score vector by rank to generate a ranked list of vector elements of the severity score vector; and
arranging the ranked list of vector elements of the severity score vector into a plurality of bins, wherein a first bin corresponds to the first severity class, and wherein the second bin corresponds to the second severity class.

3. The method of claim 1, further comprising:
selecting a portion of the second data set based on the gradient labels; and
training the second ML model or the baseline ML model via the selected portion of the second data set.

4. The method of claim 1, wherein the second data set comprises candidate biomarker data for anomalous or unhealthy set, and wherein the method further comprising:
training the second ML model or the baseline ML model via the second data set, wherein the gradient labels are used as ground truth for a set of biomarkers identified in the second data set.

5. The method of claim 1, further comprising:
outputting, via a report or display, respective gradient label and classifier output of the baseline ML model, wherein the respective gradient label and classifier output is used for diagnosis of a disease or a medical condition.

6. The method of claim 1, wherein the first data set comprises image data from a medical scan.

7. The method of claim 1, wherein the first data set comprises image data from a sensor.

8. The method of claim 1, wherein the baseline ML model comprises an auto-encoder.

9. The method of claim 4, wherein the candidate biomarker data includes at least one of:
Intraretinal Fluid (IRF), Diabetic Macular Edema (DME), and Intra-Retinal Hyper-Reflective Foci (IRHRF).

10. A method comprising:
receiving a data set;
determining, via a trained machine learning model, a presence or severity value associated with a disease or medical condition using the data set;
outputting, via a report or graphical user interface, the determined presence or severity value,
wherein the trained machine learning model was trained in a contrastive learning operation, the contrastive learning operation comprising:
training a baseline ML model via a first training data set, the first training data set consisting of data for a non-anomalous, normal, or healthy set;
generating gradient severity score vector from the baseline ML model for a second training data set, the second training data set comprising candidate biomarker data for anomalous or unhealthy set, wherein the second training data set is unlabeled with respect to severity;
tiering the severity score vector into a plurality of severity classes, including a first severity class associated with a first severity score label and a second severity class associated with a second severity score label; and
generating the trained machine learning model using the first severity score label and the second severity class.

11. The method of claim 10, wherein the step of tiering the severity score vector into the plurality of severity classes comprises:
ordering the severity score vector by rank to generate a ranked list of vector elements of the severity score vector; and
arranging the ranked list of vector elements of the severity score vector into a plurality of bins, wherein a first bin corresponds to the first severity class, and wherein the second bin corresponds to the second severity class.

12. The method of claim 10, wherein the second data set comprises candidate biomarker data for anomalous or unhealthy set, and wherein the method to train the machine learning model further comprises:
training the second ML model or the baseline ML model via the second data set, wherein the gradient labels are used as ground truth for a set of biomarkers identified in the second data set.

13. The method of claim 10, wherein the first training data set comprises image data from a medical scan.

14. The method of claim 10, wherein the first training data set comprises image data from a sensor.

15. The method of claim 10, wherein the baseline ML model comprises an auto-encoder.

16. A system comprising:
a processor; and
a memory having instructions stored thereon, wherein execution of the instructions by the processor causes the processor to:
receive a data set;
determine, via a trained machine learning model, a presence or severity value associated with a disease or medical condition using the data set;
output, via a report or graphical user interface, the determined presence or severity value,
wherein the trained machine learning model was trained in a contrastive learning operation, the contrastive learning operation comprising:
training a baseline ML model via a first training data set, the first training data set consisting of data for a non-anomalous, normal, or healthy set;
generating gradient severity score vector from the baseline ML model for a second training data set, the second training data set comprising candidate biomarker data for anomalous or unhealthy set, wherein the second training data set is unlabeled with respect to severity;
tiering the severity score vector into a plurality of severity classes, including a first severity class associated with a first severity score label and a second severity class associated with a second severity score label; and
generating the trained machine learning model using the first severity score label and the second severity score label.

17. The system of claim 16, wherein the instructions to tier the severity score vector into the plurality of severity classes comprises:
instructions to order the severity score vector by rank to generate a ranked list of vector elements of the severity score vector; and
instructions to arrange the ranked list of vector elements of the severity score vector into a plurality of bins, wherein a first bin corresponds to the first severity class, and wherein the second bin corresponds to the second severity class.

18. The system of claim 16, wherein the first training data set comprises image data from a medical scan.

19. The method of claim 16, wherein the first training data set comprises image data from a sensor.

20. The method of claim 16, wherein the baseline ML model comprises an auto-encoder.
